(19) United States
(12) Patent Application Publication
(10) Pub. No.: US 2012/0316460 A1
(43) Pub. Date: Dec. 13, 2012

(54) FLUID DELIVERY SYSTEM WITH PRESSURE MONITORING DEVICE

(76) Inventor: Christopher A. Stout, San Bruno, CA (US)

(21) Appl. No.: 13/155,350

(22) Filed: Jun. 7, 2011

(51) Int. Cl. A61B 5/03

(52) U.S. Cl. 600/561

(57) ABSTRACT

Systems and methods for determining fallopian tube occlusion are disclosed which may provide and more convenient manner of determining fallopian tube occlusion, particularly in relation to transcervical hysteroscopic sterilization with implantable inserts. In accordance with some embodiments, uterine pressure may be measured to determine occlusion with a fluid delivery system including a reservoir and a pressure monitoring device to measure a fluid pressure downstream from the reservoir.

### BACKGROUND

Embodiments of the present invention relate to the field of determining fallopian tube occlusion and in particular in relation to transcervical hysteroscopic sterilization.

Female contraception and sterilization may be effected by transervically introducing an object into a fallopian tube to inhibit conception. Devices, systems and methods for such a contraceptive approach have been described in various patents and patent applications assigned to the present assignee. For example, U.S. Pat. No. 6,526,979, U.S. Pat. No. 6,634,361, U.S. patent application Ser. No. 11/165,733 published as U.S. Publication No. 2006/0293560 and U.S. patent application Ser. No. 12/605,304 describe transcervically inserting an insert (also referred to as implant and device) into an ostium of a fallopian tube and mechanically anchoring the insert within the fallopian tube. One example of such an assembly is known as “Essure”® from Conceptus, Inc. of Mountain View, Calif. Tissue in-growth into the “Essure”® insert provides long-term contraception and/or permanent sterilization without the need for surgical procedures.

Several months after placement of the inserts within the fallopian tubes, a hysterosalpingography HSG procedure is typically utilized to determine whether the inserts have been properly positioned and whether the fallopian tubes have been occluded. During the HSG procedure a radiopaque contrast agent is injected into the uterine cavity in order to visually determine positioning of the inserts and occlusion of the fallopian tubes. HSG procedures are typically performed without controlling the injection pressure of the radiopaque contrast agent.

### SUMMARY

Embodiments of the present invention generally provide systems and methods for determining fallopian tube occlusion. In one embodiment, a dual lumen catheter fluid delivery system is described which may include a handle, a reservoir connected with the handle, an elongated shaft which houses a first and second lumens extending distally from the handle, and a pressure monitoring device to measure a fluid pressure downstream from the reservoir. For example, downstream fluid pressure may correspond to fluid pressure in the first or second lumens of fluid exiting the reservoir or fluid back pressure. The first lumen may be in operable communication with an inflatable balloon to deliver a fluid from the reservoir and into the inflatable balloon, and the second lumen may be in operable communication with an injection port distal to the inflatable balloon to deliver the fluid from the reservoir and through the injection port. The inflatable balloon may be used to form a seal against a cervix. Fluid delivered through the injection port may be used to pressurize a uterine cavity and determine whether the fallopian tubes are occluded.

A selector may be provided on the handle in order to place either the first or second lumens in operable communication with the reservoir. For example, the selector may be moveable between a first position and second position, where the first position places the first lumen (to the inflatable balloon) in operable communication with the reservoir, and the second position places the second lumen (to the injection port) in operable communication with the reservoir. In an embodiment, the reservoir has a first and second ports, and the selector is rotatable to align one of the first or second lumens with one of the first or second ports to place the first or second lumen in operable communication with the reservoir. In another embodiment, the selector comprises a manifold and a selector rod including a T-valve which can be rotated to place the first or second lumen in operable communication with the reservoir.

In accordance with embodiments of the present invention, fallopian tube occlusion may be easily and quickly determined with a hand held dual lumen catheter, where the handle and selector are sized and shaped to be gripped and operated by hand. The pressure monitoring device can measure the fluid pressure in the first lumen when the selector is in the first position. In this position, the pressure monitoring device may measure the balloon inflation pressure. The pressure monitoring device can also measure the fluid pressure in the second lumen when the selector is in the second position. In this position, the pressure monitoring device may measure the pressure in the uterine cavity. In some embodiments, the pressure monitoring device includes an analog dial display. Where the pressure monitoring device is utilized for measuring pressure in the second lumen, which corresponds to the uterine pressure, the analog dial display may include marked ranges to provide the operator with information. For example, the analog dial display can include a marked tubal occlusion pressure range, or a marked tubal perforation pressure range. The pressure monitoring device can also include a digital display. Similar to the analog dial, the digital display can include a tubal occlusion display message, or a tubal perforation display message.

A syringe may be incorporated into the handle to deliver from or store the fluid in the reservoir. In an embodiment, the handle includes a pressure syringe with a piston that is moveable in and out of the handle to reduce and expand a volume of the reservoir. For example, the piston can be moved by pushing/sliding, or by rotating/screwing the piston.

In accordance with embodiments of the present invention, a dual lumen catheter with pressure monitoring device may be operated with a modified HSG procedure. A speculum is inserted into a vagina, and the dual lumen catheter is inserted into the uterus through the cervix. The inflatable balloon of the dual lumen catheter is then inflated to hold the dual lumen catheter in place. A distention fluid is then injected through the injection port of the dual lumen catheter and into the uterine cavity as the fluid pressure is measured with the pressure monitoring device in order to determine whether the fallopian tubes have been occluded. It is not required for the distention fluid to contain a contrast agent. In one embodiment, the distention fluid is saline. Accordingly, the modified procedure may take place in an office setting, and it is not required to perform the modified procedure in an x-ray department of a hospital or large clinic.

Operation of the dual lumen catheter may be performed by manipulating a piston and selector knob to place the first and second lumens of the dual lumen catheter in operable communication with a reservoir for storing the distention fluid. In an embodiment, the dual lumen catheter may be operated by advancing the piston to reduce the volume of the reservoir and push the distention fluid through the first lumen to inflate the inflatable balloon. After sealing the cervix with the inflatable balloon the selector knob may be moved from the first position to a second position to place the reservoir in operable communication with the second lumen. Then the piston may be advanced again to reduce the volume of the reservoir and push the distention fluid through the second lumen. The operator can then monitor the fluid pressure measured by the pressure monitoring device as the distention fluid is injected into the uterine cavity. For example, the measured fluid pressure can be monitored to determine whether the pair of fallopian tubes adjacent the uterine cavity have been occluded by deposited inserts. Occlusion may be determined by both analog and digital displays on the pressure monitoring device. Where the display is analog, the occlusion may be determined where the pressure needle on the analog display maintains a constant position within a prescribed pressure range on the analog pressure gauge. Where the display is digital, the occlusion may be determined where the digital display displays a message indicating occlusion on a digital display.

In an embodiment, prior to inflating the inflatable balloon, a distal end of elongated catheter shaft may be submerged in distention fluid within a container other than the reservoir. The piston is then withdrawn to enlarge a volume of the reservoir and draw the distention fluid through the second lumen and into the reservoir. The selector is them moved to the first position to place the reservoir in operable communication with the first lumen. The piston may then be advanced to reduce the volume of the reservoir and push the distention fluid through the first lumen to inflate the inflatable balloon. In accordance with some embodiments of the invention, moving the selection knob to place the first or second lumens in operable communication with the reservoir also places the first or second lumens in operable communication with the pressure monitoring device so that the pressure monitoring device measures the fluid pressure within the lumen that is in operable communication with the reservoir. In this manner, the fluid pressure may be measured during both inflation of the inflatable balloon and during injection of the distention fluid into the uterine cavity. Alternatively, the fluid pressure is only measured during injection of the distention fluid into the uterine cavity. It is to be appreciated that fluid pressure can be measured in the first or second lumens when dispensing fluid from the reservoir as well as back pressure of the fluid in the first or second lumens when fluid is not being dispensed from the reservoir.

Embodiments of the present invention are not limited to a dual lumen catheter fluid delivery system, and other fluid delivery systems are described. In one embodiment, a multi-lumen catheter fluid delivery system is described in which, in addition to the features of the dual lumen catheter, may include a third and fourth lumens housed within the elongated shaft extending distally from the handle. The third lumen may be in operable communication with an inflatable uterine balloon, and the second and fourth lumens may be in operable communication with injection ports which are configured to be placed near left and right corneal regions of a uterine cavity upon inflation of the inflatable uterine balloon.

In accordance with other embodiments of the invention, a fluid delivery system including a reservoir and pressure monitoring device may be separate from and connectable to a conventional HSG balloon catheter or metal HSG cannula. In an embodiment, the separate fluid delivery system includes a reservoir, a fluid delivery shaft connected to the reservoir at a proximal end of the fluid delivery shaft and connected to a luer lock at a distal end of the fluid delivery shaft, a pressure monitoring device which measures the fluid pressure downstream from the reservoir, and a pressure display gauge. For example, downstream fluid pressure may correspond to fluid pressure in the fluid delivery shaft exiting the reservoir or fluid back pressure. The pressure display gauge can be analog or digital. When the pressure gauge is analog, the analog display gauge includes a marked pressure range which indicates that the fallopian tubes are occluded. When the pressure display gauge is digital, a display is provided which indicates fallopian tube occlusion on a digital display.

Fluid can be delivered from the fluid delivery system in different manners. In one embodiment, the reservoir comprises a cartridge containing pressurized fluid, which may be liquid or gas. A button may be provided which releases the pressurized fluid from the reservoir and into the fluid delivery lumen when depressed. In another embodiment, the reservoir is part of a pressure syringe in which a piston is movable to reduce and expand a volume of the reservoir. For example, the piston may be moved by pushing/sliding or rotating/screwing. In some embodiments, the fluid delivery system may further include a selector movable between first and second positions, where the first position places a first extension lumen in operable communication with the reservoir, and the second position places a second extension lumen in operable communication with the reservoir.

### BRIEF DESCRIPTION OF THE DRAWINGS

FIGS. 1A-1B illustrate isometric views of a dual lumen catheter in accordance with an embodiment of the present invention.

FIG. 1C illustrates a cross-sectional view of an elongated shaft taken along line A-A in FIG. 1A in accordance with an embodiment of the present invention.

FIG. 1D illustrates a dual lumen catheter within an expanded uterine cavity in accordance with an embodiment of the present invention.

FIG. 2A illustrates a top view of an analog dial display in accordance with an embodiment of the present invention.

FIG. 2B illustrates an isometric view of a glow in the dark analog dial display in accordance with an embodiment of the present invention.

FIG. 3A illustrates an isometric view of a handle and digital display in accordance with an embodiment of the present invention.

FIG. 3B illustrates front view of a digital display displaying a message indicating tubal occlusion in accordance with an embodiment of the present invention.

FIG. 3C illustrates front view of a digital display displaying a message tubal perforation in accordance with an embodiment of the present invention.

FIG. 4 illustrates a close-up isometric view of a pressure syringe incorporated into a dual lumen catheter in accordance with an embodiment of the present invention.

FIG. 5 illustrates a close-up isometric view of a handle in accordance with an embodiment of the present invention.

FIG. 6A illustrates a close-up side view of a selector in accordance with an embodiment of the present invention.

FIG. 6B illustrates a close-up isometric view of selector extension lumens unaligned with reservoir ports in accordance with an embodiment of the present invention.

FIG. 6C illustrates a close-up isometric view of a first selector extension lumen aligned with a first reservoir port in accordance with an embodiment of the present invention.

FIG. 6D illustrates a close-up isometric view of a second selector extension lumen aligned with a second reservoir port in accordance with an embodiment of the present invention.

FIG. 7 illustrates an isometric view of a dual lumen catheter including a selector switch in accordance with an embodiment of the present invention.

FIG. 8A illustrates a close up front view of a selector switch in a first position in accordance with an embodiment of the present invention.

FIG. 8B illustrates a close up front view of a selector switch in a second position in accordance with an embodiment of the present invention.

FIGS. 9A-9B illustrates an isometric view of a selector including selector switch and T-valve manifold assembly in accordance with an embodiment of the present invention.

FIGS. 10A-10D illustrate side views of a manner of operating a dual lumen catheter in accordance with an embodiment of the present invention.

FIG. 11A illustrates a side view of a multi-lumen catheter incorporating a uterine balloon in accordance with an embodiment of the present invention.

FIG. 11B illustrates a cross-sectional view of an elongated shaft taken along line A-A in FIG. 11 A in accordance with an embodiment of the present invention.

FIG. 11C illustrates a multi-lumen catheter incorporating an inflatable uterine balloon with within an expanded uterine cavity in accordance with an embodiment of the present invention.

FIG. 12 illustrates a side view of a fluid delivery system in accordance with an embodiment of the present invention.

FIGS. 13A-13B illustrate isometric views of a fluid delivery system in accordance with an embodiment of the present invention.

FIGS. 14A-14C illustrate isometric views of a fluid delivery system in accordance with an embodiment of the present invention.

### DETAILED DESCRIPTION

Embodiments of the present invention generally provide fluid delivery systems and manners for use thereof. More specifically, some embodiments provide fluid delivery systems with pressure monitoring devices and methods for determining fallopian tube occlusion.

Various embodiments and aspects will be described with reference to details discussed below and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present invention. In accordance with embodiments of the present invention, various fluid delivery systems described and illustrated may share substantially similar features. For clarity and conciseness, similar notation is provided in the figures where substantial similarities may exist amongst features of the various fluid delivery systems. For example, selector 114 initially described with regard to a dual lumen catheter fluid delivery system 100 may share common and substantially similar features as selectors 214 and 314 described with regard to multi-lumen catheter fluid delivery system 200 and fluid delivery system 300.

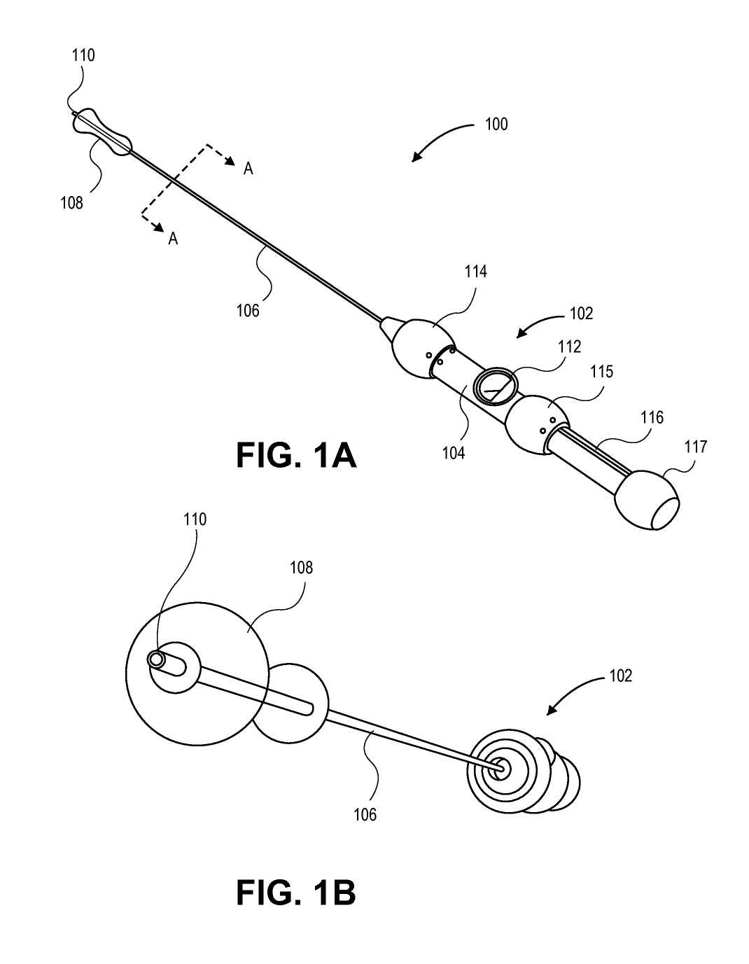

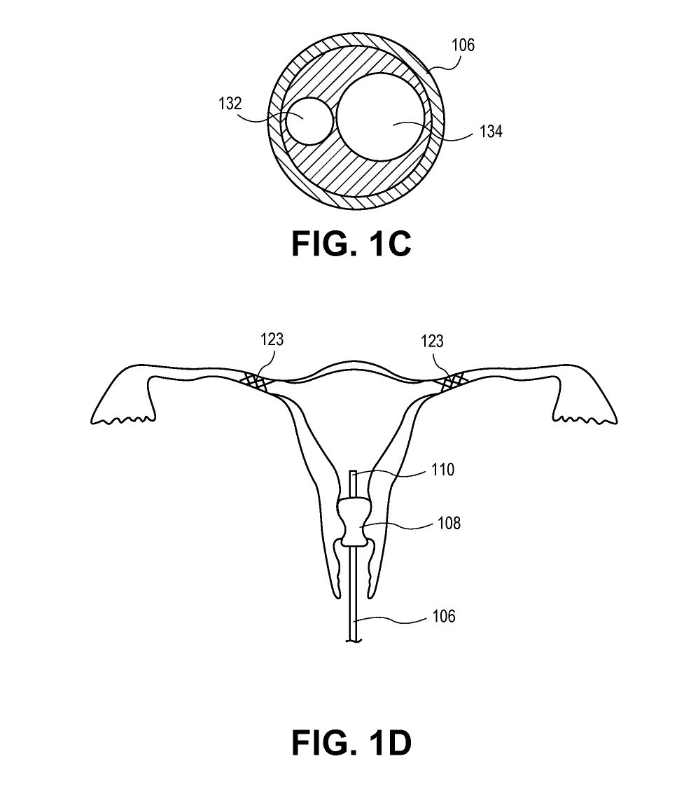

FIGS. 1A-1B illustrate isometric views of a dual lumen catheter in accordance with an embodiment of the present invention. FIG. 1C illustrates a cross-sectional view of the elongated shaft 106 taken along line A-A in FIG. 1A. As illustrated in FIGS. 1A-1C, dual lumen catheter 100 may include a handle 102, a reservoir 104 connected with or integrated into the handle, an elongated shaft 106 which houses a first and second lumens 132, 134 extending distally from the handle 102, a pressure monitoring device 112 to measure a fluid pressure downstream from the reservoir 104, and selectors 114, 115 which can be manipulated to change the operability of the dual lumen catheter 100. For example, downstream fluid pressure may correspond to fluid pressure in the first or second lumens of fluid exiting the reservoir or fluid back pressure. The handle 102 and selectors 114, 115 may be sized and shaped to be gripped and operated by hand. The first lumen 132 may be in operable communication with an inflatable balloon 108 to deliver a fluid from the reservoir and into the inflatable balloon 108. The second lumen 134 may be in operable communication with an injection port 110 distal to the inflatable balloon to deliver the fluid from the reservoir 104 and through the injection port 110. In this manner the fluid stored in the reservoir 104 of the dual lumen catheter may be used to both inflate the inflatable balloon 108 to form a seal against a cervix, and to pressurize a uterine cavity as illustrated in FIG. 1D to determine whether the adjacent fallopian tubes are occluded, for example by inserts 123 such as the Essure® insert.

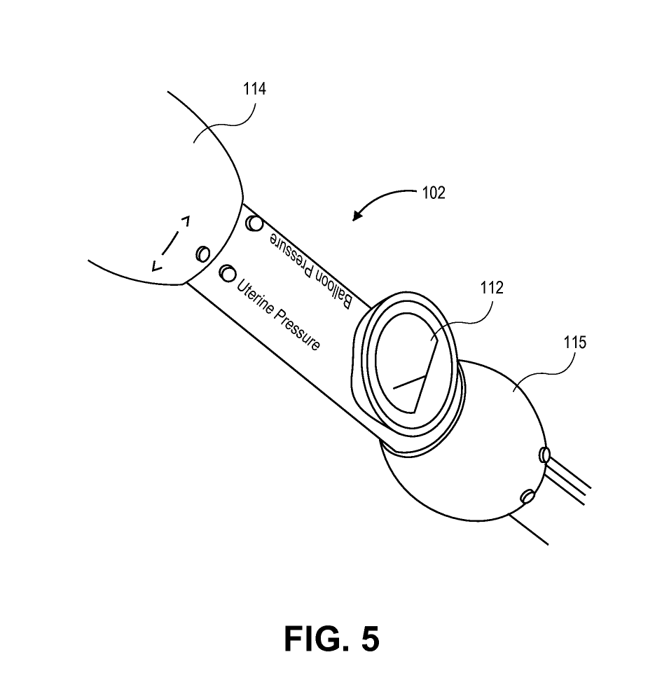

In accordance with embodiments of the present invention fallopian tube occlusion may be easily and quickly determined with the hand held dual lumen catheter, where the handle and selectors are sized and shaped to be gripped and operated by hand. As described in further detail with regard to FIGS. 5-9B, selector 114 may be provided on the handle 102 in order to place either the first or second lumen 132, 134 in operable communication with the reservoir 104. When the first lumen 132 is placed in operable communication with the reservoir 104 the pressure monitoring device 112 may measure the fluid pressure in the first lumen 132. In this position, the pressure monitoring device 11 may measure the balloon 108 inflation pressure. When the second lumen 134 is placed in operable communication with reservoir 104 the pressure monitoring device 112 may measure the fluid pressure in the second lumen 134. In this position, the pressure monitoring device 112 may measure the pressure in the uterine cavity. The measured pressure can be displayed with both analog and digital displays in accordance with embodiments of the invention. In another embodiment, the pressure monitoring device 112 only measures pressure in the second lumen 134 for measuring pressure in the uterine cavity, and the pressure monitoring device does not switch which lumen pressure is being measured when switching which lumen is in operable communication with the reservoir.

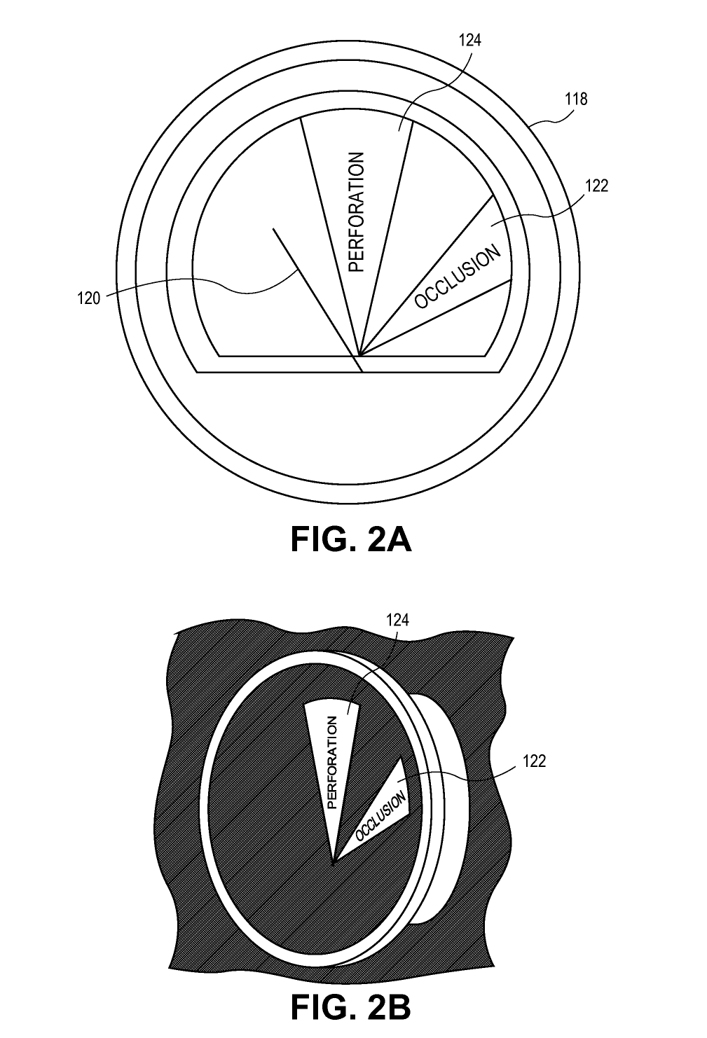

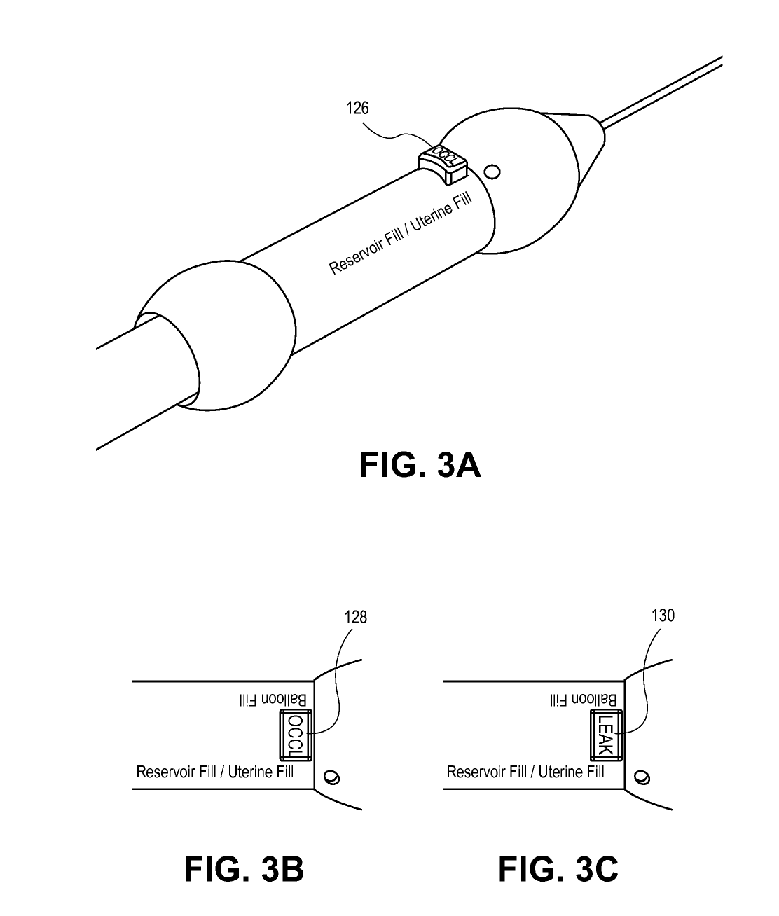

FIG. 2A is an illustration of an analog dial display 118 in accordance with an embodiment. As illustrated, the analog dial display 118 may include a needle 120 and marked pressure ranges 122, 124 to provide the operator with information. For example, the analog dial display 118 can include a marked tubal occlusion pressure range 122, or a marked tubal perforation pressure range 124. Analog dial display 118 may also include a marked pressure range corresponding to a preferred balloon inflation pressure. As illustrated in FIG. 2B, the marked pressure ranges 122, 124 may glow in the dark to accommodate use of the dual lumen catheter in a dimly lit room. As illustrated in FIGS. 3A-3C, the pressure monitoring device 112 may include a digital display 126, which depending upon the measured pressure over time can display a message 128 corresponding to tubal occlusion as illustrated in FIG. 3B, or a message 130 corresponding to tubal perforation as illustrated in FIG. 3C. Digital display 126 can also display a message corresponding to preferred balloon inflation pressure. The digital display may likewise be configured to be visible in dim lighting.

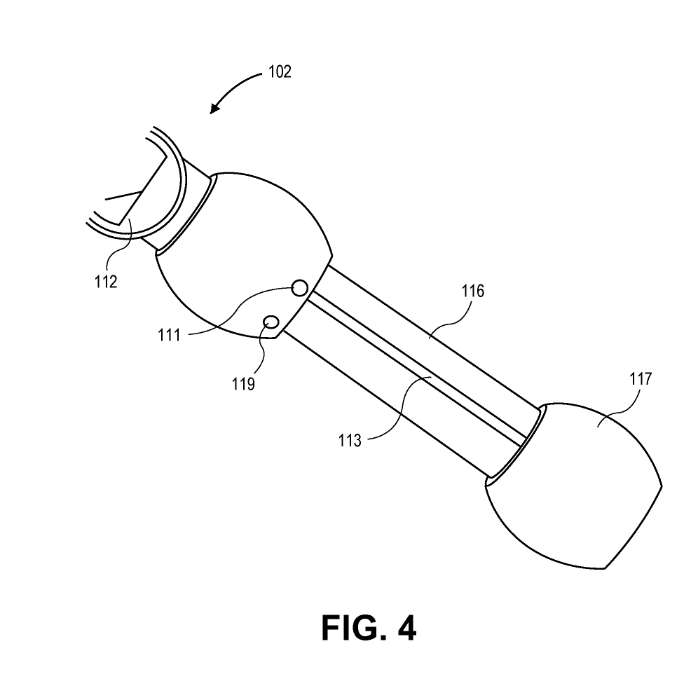

Referring now to FIG. 4 a pressure syringe may be incorporated into the dual lumen catheter to store the fluid in the reservoir and to expel the fluid from the reservoir. As illustrated, the handle 102 includes a pressure syringe with a piston 116 that is moveable in and out of the handle 102 to reduce and expand a volume of the reservoir, and consequently the amount of fluid contained within the reservoir and delivered through the first or second lumen in operable communication with the reservoir. For example, the piston 116 can be moved by pushing/sliding, by rotating/screwing the piston, or any other suitable mechanism. Piston 116 may include a knob 117 at a proximal end thereof which is sized and shaped to be gripped by hand.

A selector 115 may be included on the handle 102 in order to select from a plurality of mechanisms for moving the piston 116. As illustrated, selector 115 may be a knob which is rotatable between two positions 111, 119. In an embodiment, position 111 corresponds to a pushing or sliding mechanism for moving the piston 116, and position 119 corresponds to a rotating or screwing mechanism for moving the piston 116. In such an embodiment, pushing or sliding may be used for dispensing large quantities of fluid from the reservoir, and rotating or screwing may be used to obtain greater control on the amount of piston 116 displacement for dispensing smaller quantities of fluid from the reservoir.

Referring now to FIG. 5, a selector 114 may be provided on the handle 102 in order to place either the first or second lumen in operable communication with the reservoir. As illustrated, selector 114 may be a knob which is rotatable between first and second positions. For example, the first position may correspond to a balloon pressure position where the first lumen 132 is placed in operable communication with the reservoir so that the inflatable balloon 108 may be inflated with the fluid stored in the reservoir. The second position may correspond to a uterine pressure position where the second lumen 134 is placed in operable communication with the reservoir so that the fluid stored in the reservoir may be injected through the injection port 110 and into a uterine cavity. The second position may also correspond to a reservoir fill position where fluid can be withdrawn into injection port 110 to fill the reservoir.

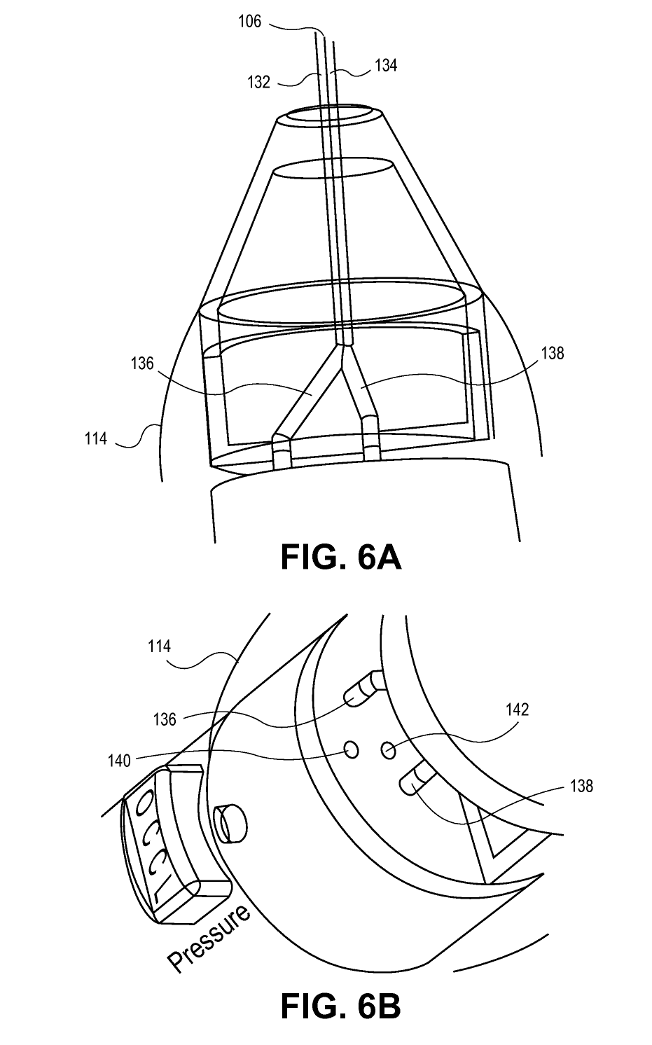

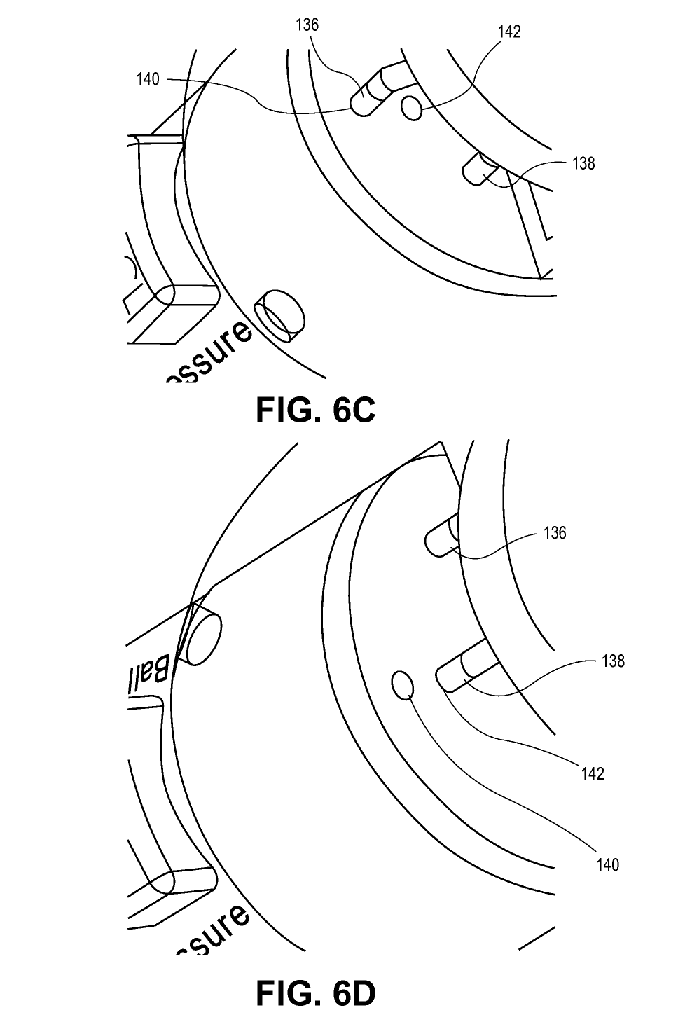

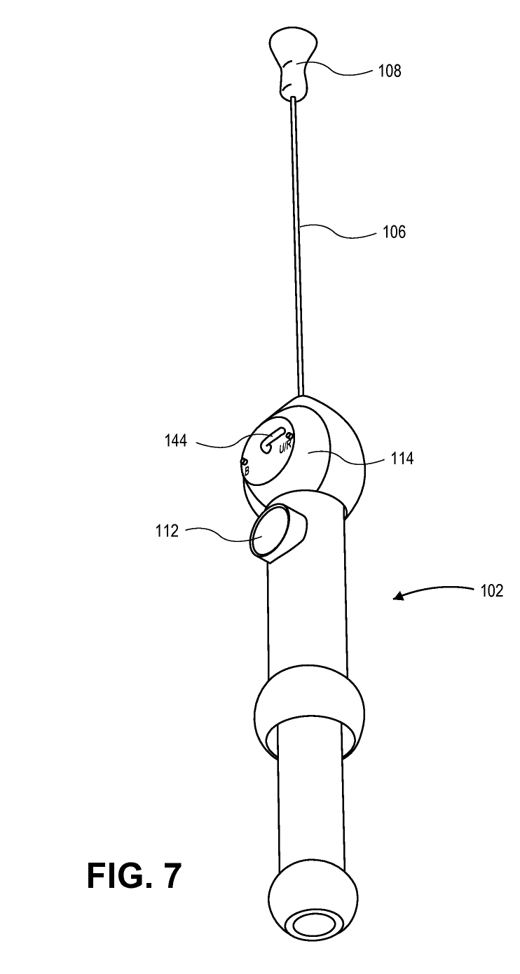

It is to be appreciated that while the foregoing description of selectors 114, 115 has been with regard to rotatable knobs, that embodiments are not limited to such and that other suitable selectors may be utilized such as, but not limited to, push buttons and switch levers. For example, FIGS. 6A-6D illustrate exemplary embodiments in which selector 114 includes a rotatable knob, and FIGS. 7-9B illustrate exemplary embodiments in which selector includes a switch lever.

Referring now to FIG. 6A, a selector 114 is illustrated as including a rotatable knob which houses a first and second extension lumens 136, 138 which are connected to the first and second lumens 132, 134 that extend through the elongated shaft 106. The first and second extension lumens 136, 138 are configured so that the selector 114 can be rotated between a first position where the first extension lumen 136 aligns with a first reservoir port 140, and a second position where the second extension lumen 138 aligns with a second reservoir port 142. FIG. 6B is an illustration of the relative positions of the first and second extension lumens 136, 138 and first and second reservoir ports 140 with the selector at an intermediate position between the first and second positions. In this intermediate position, neither of the first or second extension lumens 136, 138 is in alignment with either of the first or second reservoir ports 140, 142. FIG. 6C is an illustration of the relative positions of the first and second extension lumens 136, 138 and first and second reservoir ports 140, 142 with the selector at the first position. As illustrated, in the first position the first extension lumen 136 is in alignment with the first reservoir port 140, while the second extension lumen 138 and second reservoir port 142 are misaligned. In the first position, the first lumen 132 (to the inflatable balloon 108) is in operable communication with the reservoir. FIG. 6D is an illustration of the relative positions of the first and second extension lumens 136, 138 and first and second reservoir ports 140, 142 with the selector at the second position. As illustrated, in the second position the second extension lumen 138 is in alignment with the second reservoir port 142, while the first extension lumen 136 and first reservoir port 140 are misaligned. In the second position, the second lumen 134 (to the injection port 110) is in operable communication with the reservoir.

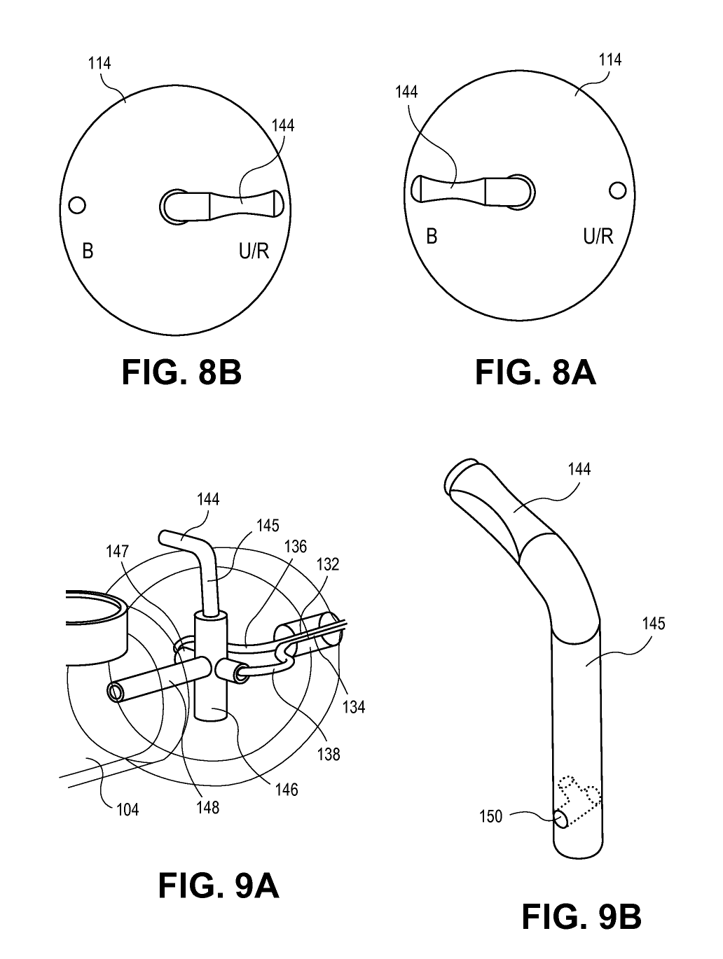

Referring now to FIG. 7 and FIGS. 8A-8B, a selector 114 may include a switch lever 144, which can be moved between first position illustrated in FIG. 8A which places the first lumen (to the inflatable balloon) in operable communication with the reservoir, and a second position illustrated in FIG. 8B which places the second lumen (to the injection port) in operable communication with the reservoir. In an embodiment, operation of the switch lever 144 may be accomplished with a T-valve manifold assembly as illustrated in FIGS. 9A-9B. As illustrated, switch lever 144 may extend from a selector rod 145 including a T-valve port 150 which can be rotated between the first and second positions to place either the first or second lumens 132, 134 in operable communication with the reservoir 104. Referring to FIG. 9A, a shaft 148 extends from the reservoir to a T-valve manifold 146. First and second extension lumens 136, 138 are connected at their distal ends to lumens 132, 134 and at their proximal ends to first and second T-valve manifold ports 147, 149. FIG. 9B is an illustration of switch lever 144 in the first position, in which the T-valve port 150 is positioned to place the first lumen 132 in operable communication with the reservoir 104. When the switch lever 144 is moved to the second position, the selector rod 145 may rotate approximately 90 degrees such that the T-valve port 150 is positioned to place the second lumen 134 in operable communication with the reservoir 104.

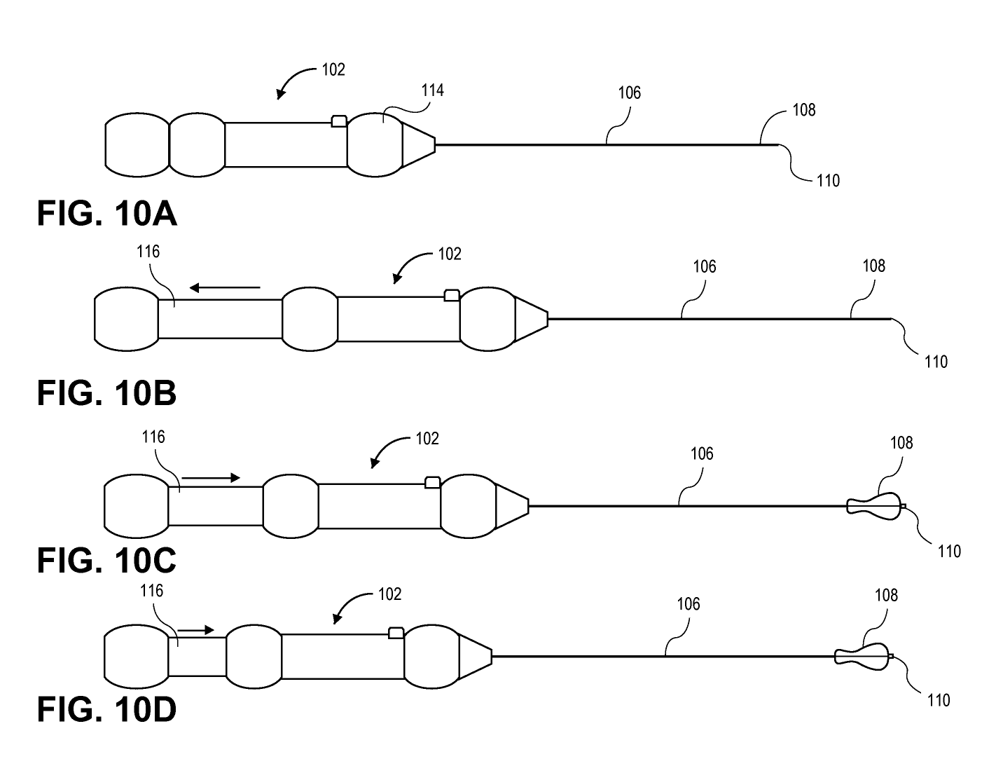

Referring now to FIGS. 10A-10D a manner of operating the dual lumen catheter is described in accordance with an embodiment of the present invention. Prior to inflating the balloon 108 the distal end comprising injection port 110 of the elongated catheter shaft 106 may be first submerged in distention fluid within a container (not illustrated) other than the reservoir. With the selection knob 114 in the second position, piston 116 may then be withdrawn as illustrated in FIG. 10B to enlarge a volume of the reservoir and draw the distention fluid through the second lumen 134 and into the reservoir. The selection knob 114 may then be moved to the first position to place the reservoir in operable communication with the first lumen 132. With the reservoir filled with distention fluid, the distal end of the dual lumen catheter may then be inserted into the uterine cavity through the cervix. With the selection knob 114 now in the first position, piston 114 may be advanced as illustrated in FIG. 10C to reduce the volume of the reservoir and push the distention fluid through the first lumen 132 to inflate the balloon 108 and seal the cervix. The operator may monitor the fluid pressure being measured by the pressure monitoring device while advancing the piston 114 to monitor the balloon 108 inflation pressure. After sealing the cervix with the inflatable balloon 108 the selector knob 114 may be moved from the first position to the second position to place the reservoir in operable communication with the second lumen 134. The operator can then monitor the fluid pressure measured by the pressure monitoring device while advancing the piston 114 as illustrated in FIG. 10D to reduce to the volume of the reservoir and inject the distention fluid into and pressurize the uterine cavity, as illustrated in FIG. 1D. In an embodiment, the fluid pressure is monitored while pressurizing the uterine cavity to determine whether the pair of fallopian tubes adjacent the uterine cavity have been occluded, for example by deposited inserts such as the Essure® inserts. Tubal occlusion may be determined by both analog and digital displays on the pressure monitoring device.

In an embodiment, where the display is analog, tubal occlusion may be determined by injecting the distention fluid into the uterine cavity until the pressure needle 120 on the analog display 118 maintains a constant position within the prescribed tubal occlusion pressure range 122 on the analog display 118 described with regard to FIG. 2. During initial injection of fluid into the uterine cavity a spike in fluid pressure within the respective lumen being measured may be observed. Upon stopping injecting of fluid, the pressure monitoring device may measure the back pressure of fluid from the uterine cavity into the respective lumen being measured. If the uterine cavity is not distended then the back pressure may be low. For example, in an embodiment the back pressure can be between 0 mm Hg and 25 mm Hg for a uterus which is not distended. If the back pressure is decreasing or approximately constant in a range 124 lower than the tubal occlusion range 122, then this may be an indication that the uterine tissue is absorbing the distention fluid or that the uterine muscle is stretching into a larger distended shape. Decreasing pressure or an approximately constant pressure in a range 124 lower than the tubal occlusion range 122 may also be an indication of perfusion such as a leak in the cervical seal with the balloon 108, a leak down one of the fallopian tubes, or a perforation in the uterus or cervix. For example, a perfusion range 124 may be between 25 mm Hg and 75 mm Hg in an embodiment. In accordance with embodiments of the present invention, observation of an approximately constant higher pressure in a pressure range 122 such as 150 mm Hg to 250 mm Hg may indicate tubal occlusion. It is to be appreciated that an operator may avoid extremely high pressures to ensure that inserts 123 are not moved from their intended locations, and to avoid patient discomfort.

In an embodiment, where the display is digital, a digital display 126 as illustrated in FIGS. 3A-3C may display a message 128 corresponding to tubal occlusion if an approximately constant high pressure within the previously described tubal occlusion pressure range is measured, and a message 130 corresponding to a system leak may be displayed if a decreasing or approximately constant pressure in a pressure range lower than the previously described tubal occlusion pressure range is measured.

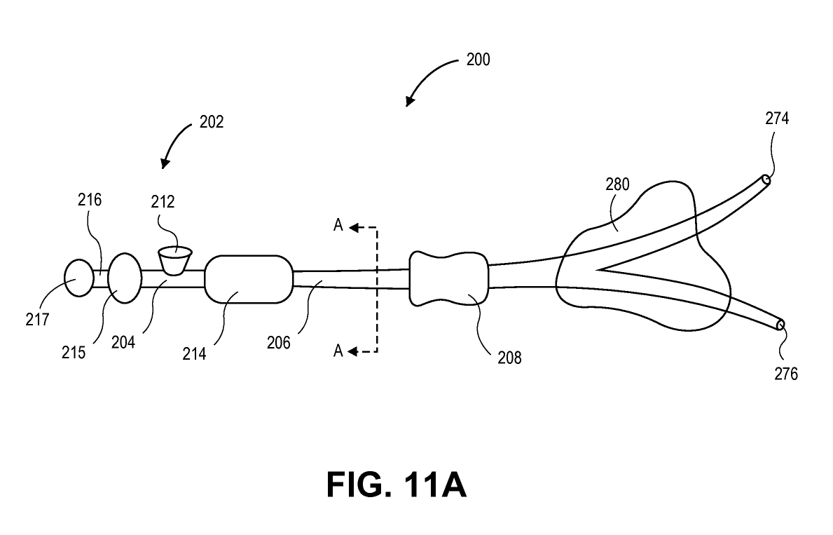

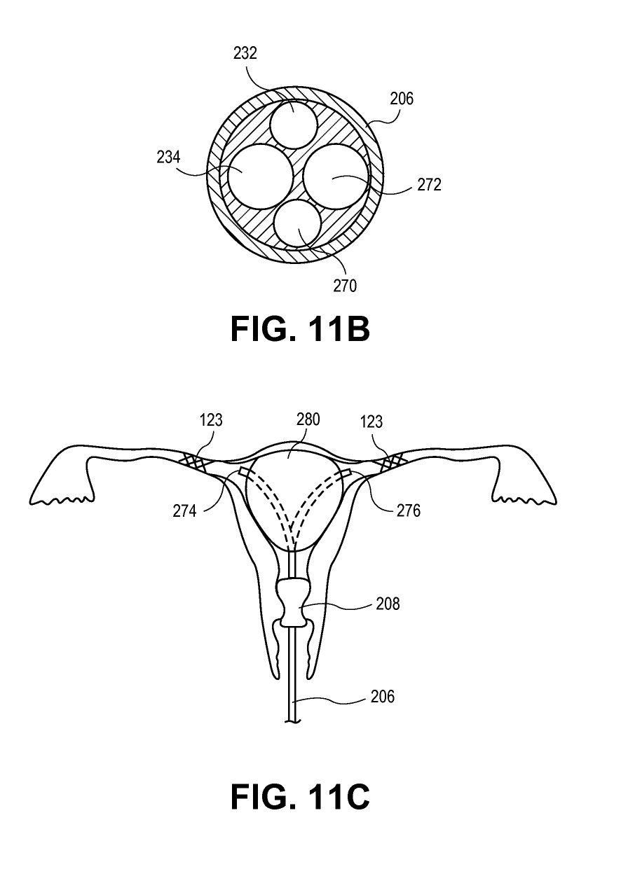

In another embodiment, tubal occlusion may be determined utilizing a multi-lumen catheter incorporating a uterine balloon. FIGS. 11A-11C illustrate a multi-lumen catheter with substantial similarities to the dual lumen catheter described above. As illustrated, the multi-lumen catheter 200 may include a handle 202, a reservoir 204 connected with or integrated into the handle, a pressure syringe including a piston 216 and knob 217, a forked elongated shaft 206 which houses multiple lumens extending distally from the handle 202, a pressure monitoring device 212 to measure fluid pressure downstream from the reservoir 204, and selectors 214, 215 which can be manipulated to change the operability of the multi-lumen catheter 200. For example, downstream fluid pressure may correspond to fluid pressure in one of the multiple lumens of fluid exiting the reservoir or fluid back pressure. The handle 202 and selectors 214, 215 may be sized and shaped to be gripped and operated by hand. A first lumen 232 may be in operable communication with an inflatable balloon 208 to deliver a fluid from the reservoir and into the inflatable balloon 208. A second lumen 234 may be in operable communication with an injection port 274 distal to the inflatable uterine balloon 280 to deliver the fluid form the reservoir 204, through the injection port 274 and into a left fallopian tube. A third lumen 270 may be in operable communication with an inflatable uterine balloon 280 to deliver a fluid from the reservoir and into the inflatable uterine balloon 280. A fourth lumen 272 may be in operable communication with an injection port 276 distal to the inflatable uterine balloon 280 to deliver the fluid from the reservoir 204, through the injection port 276 and into a right fallopian tube. In this manner, as illustrated in FIG. 11C, the fluid stored in the reservoir 204 of the multi-lumen catheter may be used to both inflate the inflatable balloon 208, to seal the cervical canal, inflate the inflatable uterine balloon 280 to seal the corneal regions of the uterine cavity, and to pressurize the cornual regions of the uterine cavity adjacent the fallopian tubes to determine whether a specific fallopian tube adjacent an injection port 274, 276 is occluded, for example by inserts 123 such as the Essure® insert.

In accordance with embodiments of the invention, pressure monitoring device 212 may operate similarly as pressure monitoring device 112 previously described. Likewise, selector 215 may operate similarly as selector 115 previously described, and selector 214 may operate similarly as selector 114 with any necessary modifications to accommodate additional lumens.

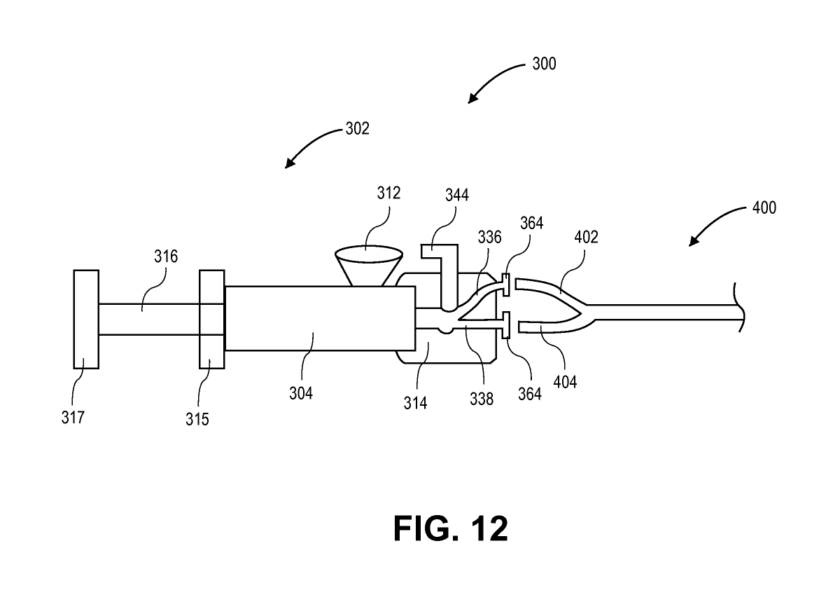

In other embodiments of the present invention, tubal occlusion may be determined utilizing a fluid delivery system, such as those illustrated in FIGS. 12-14C, which can be connected to conventional balloon HSG catheters or metal HSG cannulas. Referring to FIG. 12, in an embodiment the handle 302 of a fluid delivery system 300 has substantial similarities to the handle 102 of the dual lumen catheter 100 described above. As illustrated, a reservoir 304 and pressure syringe including a piston 316 and knob 317 may be connected with or integrated into the handle 302. A pressure monitoring device 312 is provided to measure fluid pressure downstream from the reservoir 304. Selectors 314, 315 may be sized and shaped to be gripped and operated by hand, and manipulated to change the operability of the fluid delivery system 300. Selector 314 may be provided on the handle 302 and moveable between a first and second positions in order to place either a first extension lumen 336 or a second extension lumen 338 in operable communication with the reservoir 304. In the first position, the first extension lumen 336 is placed in operable communication with the reservoir 304 and the pressure monitoring device 312 may measure pressure in the first extension lumen 336. In the second position, the second extension lumen 338 is placed in operable communication with the reservoir 304 and the pressure monitoring device 312 may measure pressure in the second extension lumen 338. Luer locks 364 may be placed on the distal ends of extension lumens 336, 338 in order to connect with luer channels 402, 404 on a separate balloon HSG catheter 400 or metal HSG cannula. In this manner luer locks 364 of fluid delivery system 300 are connected to luer channels 402, 404 of a separate balloon HSG catheter 400 or metal HSG cannula. When selector 314 is in the first position, the reservoir 304 may be in operable communication with a balloon of the HSG catheter 400 and pressure monitoring device 312 may measure the balloon inflation pressure, and when selector 314 is in the second position, the reservoir 304 may be in operable communication with a injection port of the HSG catheter 400 and the pressure monitoring device 312 may measure the pressure in the uterine cavity. The measured pressure can be displayed with both analog and digital displays in accordance with embodiments of the invention.

Selector 314 may be any suitable selector in accordance with embodiments of the invention. For example, selector 314 comprise a rotatable selector knob similar to that described with regard to FIGS. 5-6D, or selector 314 may comprise a switch lever 344 similar to that described with regard to FIGS. 7-9B.

A selector 315 may be included on the handle 302 in order to select from a plurality of mechanisms for moving a piston 316. Similar to that described with regard to FIG. 4, selector 315 may be a knob which is rotatable between two positions. In an embodiment, one position corresponds to a pushing or sliding mechanism for moving the piston 316, and another position corresponds to a rotating or screwing mechanism for moving the piston 116. In such an embodiment, pushing or sliding may be used for dispensing large quantities of fluid from the reservoir, and rotating or screwing may be used to obtain greater control on the amount of piston 316 displacement for dispensing smaller quantities of fluid from the reservoir.

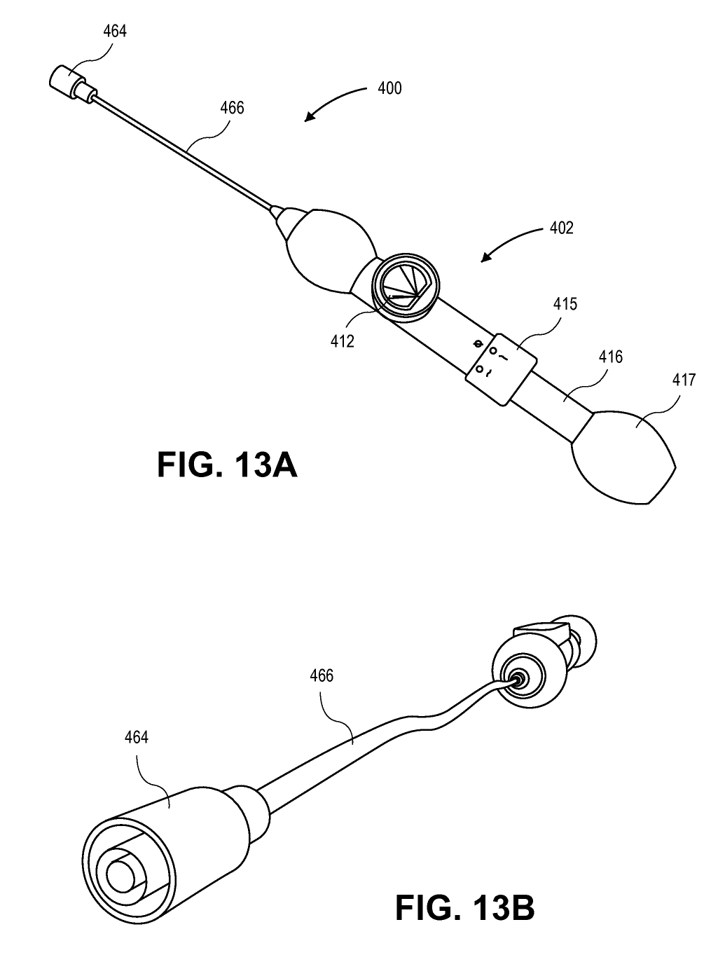

Referring now to FIGS. 13A-13B, in an embodiment, tubal occlusion may be determined utilizing a fluid delivery system 400 including a single lumen within elongated shaft 466 which can be connected to a conventional balloon HSG catheter or metal HSG cannula with a luer lock 464. Fluid delivery system 400 may be substantially similar to the dual lumen catheter described above with one difference being the fluid delivery system 400 comprises a single lumen catheter rather than a dual lumen catheter, and the fluid delivery system 400 does not include a selector 114. In this manner, pressure monitoring device 412 measures the fluid pressure in the single lumen exiting the reservoir. The single lumen may be placed in operable communication a luer channel of a conventional balloon HSG catheter or metal HSG cannula to measure the pressure in the channel, which may be connected to an inflatable balloon or uterine cavity, for example. Similar to the dual lumen catheter describe above, the fluid delivery system 400 may also incorporate a selector 415 and syringe including a piston 416 and knob 417 into handle 402.

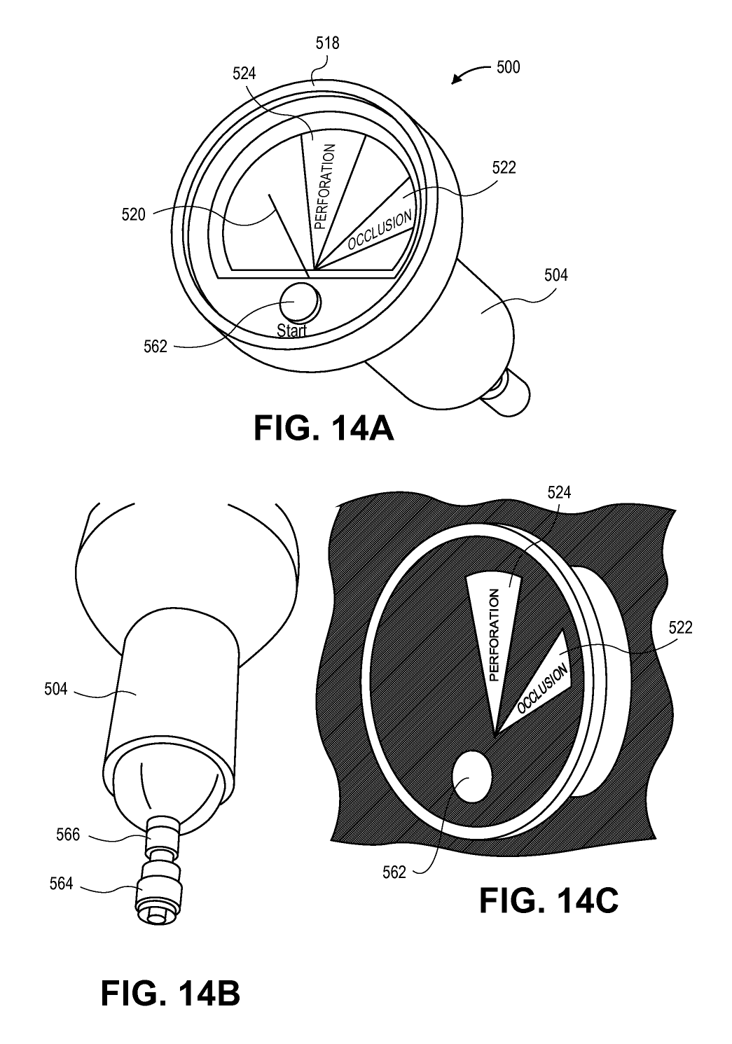

Referring now to FIGS. 14A-14C, in an embodiment, tubal occlusion may be determined utilizing a fluid delivery system 500 including a single lumen within a shaft 566 which can be connected to a conventional balloon HSG catheter or metal HSG cannula with a luer lock 564. Fluid delivery system 500 may include a pressurized reservoir 504 storing a fluid and an analog dial display 518 in accordance with an embodiment. In an embodiment, the pressurized reservoir 504 includes a cartridge containing the pressurized fluid. The analog dial display 518 may include a needle 520 and marked pressure ranges 522, 524 to provide the operator with information. For example, the analog dial display 518 can include a marked tubal occlusion pressure range 522, or a marked tubal perforation pressure range 524. Alternatively, fluid delivery system 500 may include a digital display similar to that described with regard to FIGS. 3A-3C. Fluid delivery system 500 may additionally include a button 562 to dispense the fluid from the pressurized reservoir 504. Similar to FIG. 2B, the marked ranges 522, 524 and button 562 of analog dial display 518 may glow in the dark to accommodate use of the fluid delivery system 500 in a dimly lit room. In use, an operator may hold fluid delivery system 500 by hand and press button 562 with the same hand to dispense the pressurized fluid from the reservoir and into a uterine cavity or balloon while monitoring the pressure reading as discussed above.

In the foregoing specification, various embodiments of the invention have been described. It will, however, be evident that various modifications and changes may be made thereto without departing from the broader spirit and scope of the invention as set forth in the appended claims. The specification and drawings are, accordingly, to be regarded in an illustrative sense rather than a restrictive sense. Hence, the scope of the present invention is limited solely by the following claims.

What is claimed is:

1. A fluid delivery system comprising:
a handle;
a reservoir connected with the handle;
an elongated shaft housing a first and second lumens extending distally from the handle;
wherein the first lumen is in operable communication with an inflatable balloon to deliver a fluid from the reservoir and into the inflatable balloon, and the second lumen is in operable communication with an injection port distal to the inflatable balloon to deliver the fluid from the reservoir and through the injection port; and
a pressure monitoring device to measure a fluid pressure downstream from the reservoir.

2. The fluid delivery system of claim 1, wherein the handle further comprises a selector moveable between a first and second positions, wherein the first position places the first lumen in operable communication with the reservoir, and the second position places the second lumen in operable communication with the reservoir.

3. The fluid delivery system of claim 2, wherein the handle and selector are sized and shaped to be gripped and operated by hand.

4. The fluid delivery system of claim 1, wherein the pressure monitoring device measures the fluid pressure in the first lumen when the selector is in the first position, and the pressure monitoring device measures the fluid pressure in the second lumen when the selector is in the second position.

5. The fluid delivery system of claim 1, wherein the handle includes a pressure syringe.

6. The fluid delivery system of claim 5, wherein the pressure syringe comprises a piston which is moveable in and out of the handle to reduce and expand a volume of the reservoir.

7. The fluid delivery system of claim 6, wherein the piston is moved in and out of the handle by sliding or rotating the piston.

8. The fluid delivery system of claim 1, wherein the pressure monitoring device includes an analog dial display.

9. The fluid delivery system of claim 8, wherein the analog dial display comprises a marked tubal occlusion pressure range.

10. The fluid delivery system of claim 9, wherein the analog dial display comprises a marked tubal perforation pressure range.

11. The fluid delivery system of claim 1, wherein the pressure monitoring device comprises a digital display.

12. The fluid delivery system of claim 11, wherein the digital display comprises a tubal occlusion display message.

13. The fluid delivery system of claim 11, wherein the digital display comprises a tubal perforation display message.

14. The fluid delivery system of claim 2, wherein the reservoir has a first and second ports, and the selector is rotatable to align one of the first or second lumens with one of the first or second ports to place the first or second lumen in operable communication with the reservoir.

15. The fluid delivery system of claim 2, wherein the selector comprises a manifold and a selector rod including a first and second ports which can be rotated to place the first or second lumen in operable communication with the reservoir.

16. The fluid delivery system of claim 2, further comprising:
a third and fourth lumens housed within the elongated shaft extending distally from the handle;
wherein the third lumen is in operable communication with an inflatable uterine balloon; and
wherein the second and fourth lumens are in operable communication with injection ports which are configured to be placed near left and right corneal regions of a uterine cavity upon inflation of the inflatable uterine balloon.

17. A method of operating a fluid delivery system comprising:
advancing a piston to reduce a volume of a reservoir and push a distention fluid through a first lumen to inflate an inflatable balloon;
moving a selector knob from a first position to a second position to place the reservoir in operable communication with a second lumen; and
advancing the piston to reduce the volume of the reservoir and push the distention fluid through the second lumen.

18. The method of claim 17, further comprising:
prior to inflating the inflatable balloon:
submersing a distal end of an elongated catheter shaft in the distention fluid;
withdrawing the piston to enlarge a volume of the reservoir and draw the distention fluid through the second lumen and into the reservoir; and
moving the selector to the first position to place the reservoir in operable communication with the first lumen.

19. The method of claim 17, wherein the distention fluid consists of saline.

20. The method of claim 17, wherein the distention fluid does not comprise a contrast agent.

21. The method of claim 17, further comprising inflating the inflatable balloon against a cervical canal.

22. The method of claim 21, further comprising measuring a fluid pressure of the distention fluid while pushing the distention fluid through the second lumen.

23. The method of claim 22, further comprising measuring the fluid pressure of the distention fluid to determine whether a pair of fallopian tubes are occluded by a pair of inserts.

24. The method of claim 23, wherein determining occlusion comprises maintaining a pressure needle at a constant position on an analog pressure gauge.

25. The method of claim 23, wherein determining occlusion comprises displaying a message indicating occlusion on a digital display.

26. A fluid delivery system comprising:
a reservoir;
a fluid delivery shaft connected to the reservoir at a proximal end of the fluid delivery shaft, and connected to a luer lock at a distal end of the fluid delivery shaft;
a pressure monitoring device which measures a fluid pressure downstream from the reservoir; and
a pressure display gauge selected from the group consisting of:
an analog display gauge which includes a marked pressure range which indicates fallopian tube occlusion; and
a digital display gauge which displays a message indicating fallopian tube occlusion on a digital display.

27. The fluid delivery system of claim 26, wherein the reservoir comprises a cartridge containing a pressurized fluid.

28. The fluid delivery system of claim 27, wherein the pressurized fluid is a gas.

29. The fluid delivery system of claim 27, further comprising a button which releases the pressurized fluid from the reservoir and into the fluid delivery shaft when depressed.

30. The fluid delivery system of claim 26, further comprising a pressure syringe in which a piston is movable to reduce and expand a volume of the reservoir.

31. The fluid delivery system of claim 30, wherein the piston is moved by sliding or rotating the piston.

32. The fluid delivery system of claim 26, further comprising a selector moveable between a first and second positions, wherein the first position places a first extension lumen in operable communication with the reservoir, and the second position places a second extension lumen in operable communication with the reservoir.
